(19) United States
(12) Patent Application Publication
(10) Pub. No.: US 2010/0274104 A1
(43) Pub. Date: Oct. 28, 2010

(54) INFANT/CHILD MONITOR

(76) Inventor: Sitara R. Khan, Old Westbury, NY (US)

Correspondence Address: Thomas A. O'Rourke;Bodner & O'Rourke, 425 Broadhollow Road, Melville, NY, 11747, US

(21) Appl. No.: 12/734,107

(22) PCT Filed: Oct. 9, 2008
(86) PCT No.: PCT/US08/79433

§ 371 Date: Jun. 28, 2010

Related U.S. Application Data
(60) Provisional application No. 60/978,731, filed on Oct. 9, 2007.

(51) Int. Cl. A61B 5/00

(52) U.S. Cl. 600/301

(57) ABSTRACT

A monitoring device including a plurality of sensors, each sensor configured to measure one of a plurality of vital sign parameters and a plurality of environmental parameters. The monitoring device further including a memory configured to store at least a programmable upper limit and a programmable lower limit for each of the measured parameters, a central processing unit configured to process the measured parameters and compare each measured parameter against its respective programmable upper limit and programmable lower limit, and at least one communications module configured to transmit and receive data. Further, the monitoring device being configured to automatically transmit an alert when one measured parameter is outside a range defined by its respective programmable upper limit and programmable lower limit. Optionally, the device includes various communications interfaces, including cellular, Internet, conventional telephone. Other optional features include an emergency dialer, positioning technology such as GPS, a microphone, and a speaker.

### CROSS REFERENCE TO RELATED APPLICATIONS

This application claims priority to U.S. Provisional Application Ser. No. 60/978,731, filed Oct. 9, 2007, entitled “Infant/Child Monitor.” The contents of which are incorporated herein by reference in its entirety.

### FIELD OF THE INVENTION

The present invention generally relates to monitoring vital functions of infants, children, adults and the elderly, sensing location and alerting emergency contacts.

### BACKGROUND

Being a caretaker can be a difficult job. The health of those being looked after includes monitoring of various health parameters associated with the subject being taken care of. When that subject is an infant there is a risk of Sudden Infant Death Syndrome (or SIDS), which is a fatal condition in which a sleeping infant stops breathing. Some children are at a greater risk from SIDS than others. Parents who are aware of greater risk factors in their children, worry each time they put their baby down to sleep.

When the subject being taken care of is a child or elderly adult, these subjects also need monitoring as well. However, having the caretaker constantly monitoring all of the parameters of the subjects in person by manual methods can be a difficult and time consuming process, if not impossible. This would require the caretaker to be with the subject continuously to take measurements and monitor various internal and environmental parameters. Thus, there is a need for an automated system of monitoring these parameters and altering the caretaker when there is a problem.

### SUMMARY

An embodiment of the present invention provides a monitoring device including a plurality of sensors, each sensor configured to measure one of a plurality of vital sign parameters and a plurality of environmental parameters. The monitoring device further includes a memory configured to store at least a programmable upper limit and a programmable lower limit for each of the measured parameters, a central processing unit configured to process the measured parameters and compare each measured parameter against its respective programmable upper limit and programmable lower limit, and at least one communications module configured to transmit and receive data. Further, the monitoring device is configured to automatically transmit an alert when one measured parameter is outside a range defined by its respective programmable upper limit and programmable lower limit.

### BRIEF DESCRIPTION OF THE DRAWINGS

The foregoing and other features of the present invention will be more readily apparent from the following detailed description and drawings of illustrative embodiments of the invention in which:

FIG. 1 is an illustration of a monitoring device in accordance with an embodiment of the present invention;

FIG. 2 is a block diagram of a monitoring system in accordance with an embodiment of the present invention;

FIG. 3 is an illustration of a monitoring device in accordance with an embodiment of the present invention;

FIG. 4 is an illustration of a peripheral mobile device in accordance with an embodiment of the present invention;

FIG. 5 is an illustration of a base unit in accordance with an embodiment of the present invention;

FIG. 6 is an illustration of a medication box in accordance with an embodiment of the present invention; and

FIG. 7 is an illustration of a monitoring device in accordance with an embodiment of the present invention.

### DETAILED DESCRIPTION

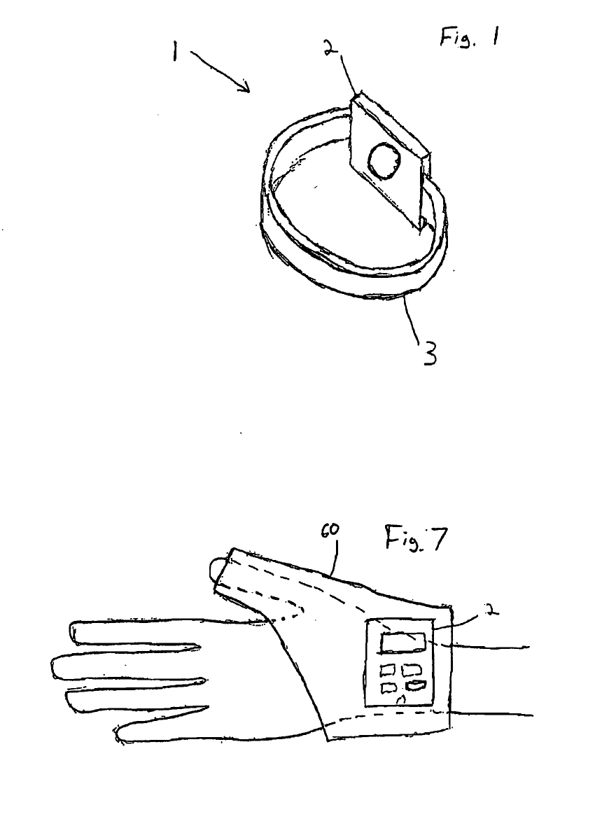

FIG. 1 shows a monitoring device 1 according to an embodiment of the present invention. The monitoring device 1 includes a monitoring unit 2 coupled to a band 3. Although the band 3 is preferably made of an elastic material, it can be made of any material that can secure the monitoring unit 2 to a user. The band 3 is sized to fit over the wrist of the user, typically an infant, child, adult, or elderly person. The band 3 is preferably secured on the wrist of the user, and creates a secure fit so that vital functions may be monitored effectively without overly restricting the flow of blood. While it is preferable to monitor vital functions at the wrist of a user, the monitoring unit 2 may be attached to an infant by any means and at any site suitable for monitoring vital functions, including the ankle.

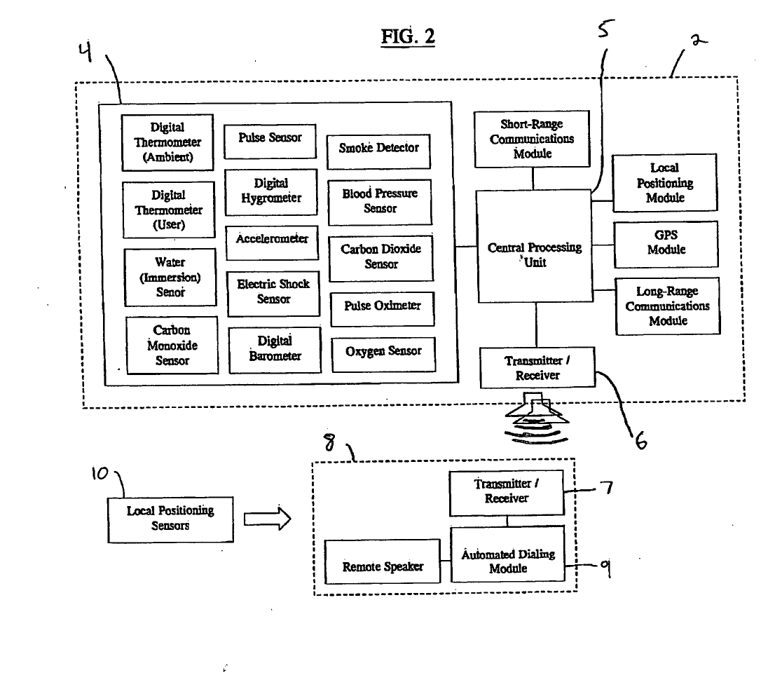

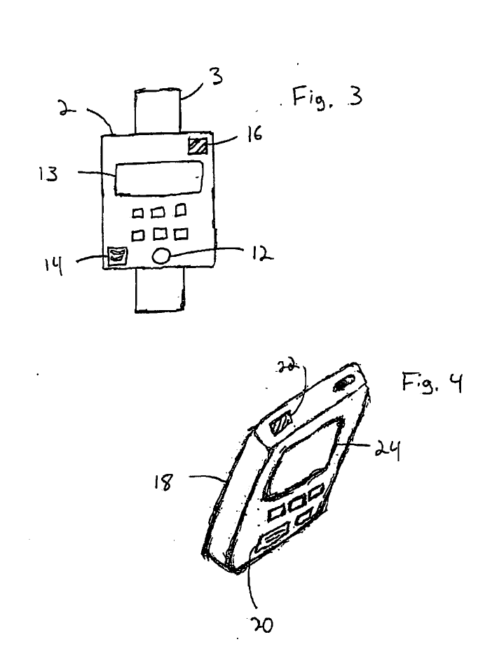

FIG. 2 is a block diagram of the monitoring unit 2 according to an embodiment of the present invention. The monitoring unit 2 includes multiple sensors 4 and a central processing unit 5. The sensors 4 measure various vital signs associated with the user, and environmental parameters in the user's proximity. The sensors 4 are in continuous communication with the central processing unit 5. The central processing unit 5 includes a microprocessor, memory, and suitable programming for operating the device. Each of the sensors 4 monitors and measures a specific parameter, and transmits the parameter to the central processing unit 5. The central processing unit 5 compares the measured parameter against a predetermined range which is deemed healthy or safe. Thus, the sensor input can be checked against upper limits and lower limits for vital signs, such as upper and lower limits for blood pressure, temperature, oxygen, pulse, etc. In this way, the user can be monitored for a number of distress conditions, and an alert can be transmitted if a measured parameter falls outside the predetermined range, indicating a distress condition.

The sensors 4 included in the monitoring unit 2 may vary based upon which vital functions are to be monitored. For example, it may be desirable to sense an infant's blood pressure (measured by a sphygmomanometer), pulse rate, temperature, and oxygen saturation (measured by a pulse oximeter). Monitoring the vital signs of the infant can aid in the prevention of sudden infant death syndrome (SIDS).

Additionally, the sensors 4 may include an accelerometer to sense any sudden jolting or jarring motion of the user. Accordingly, an alert is transmitted if the user receives a sudden jolt or is shaken vigorously, thus recognizing seizures, falls or any vigorous and violent shaking of the child or infant by an abuser, triggering the accelerometer. Further, these sudden motions are characteristic of shaken baby syndrome, which is estimated to cause 1,200 to 1,600 acceleration-deceleration injuries in infants each year. The accelerometer communicates with the central processing unit 5 via an electrical signal, the strength of which will be checked against a predetermined safe range over a period of time, thereby detecting whether there has been an unsafe specific external force or disparate changes in the same.

Other sensors may be incorporated to monitor the external environment of the user, such as air temperature, pressure, humidity and air quality. Digital thermometers, barometers, hygrometers and various air quality sensors (such as carbon monoxide and smoke detectors) may be used for such purposes respectively.

The monitoring unit 2 can come preprogrammed with factory presets for acceptable limits for the sensed parameters (i.e., temperature, pulse, blood pressure, etc.). However, these acceptable limits can also be adjusted to suit the individual user. For example, if the person who is being monitored by the device has a normally low pulse rate or body temperature, the device can be adjusted so that the upper and lower limits are set based on the individual's natural normal vital sign parameters.

When a measured value from the sensors 4 lies outside the predetermined range, thereby indicating that there is a problem and the user is in distress, the central processing unit 5 transmits a signal via a transmitter 6. The transmitted signal may be in the form of an alert, may include the measured value and the predetermined range, or may be in any form that is helpful to an emergency responder or health-care professional. The signal is received by a receiver 7 at the dialing station 8. The signal is then communicated to an automated dialing module 9. The dialing station 8 includes a processor which transmits information to stored emergency contact numbers. The information may be an alert or other data associated with the user and the user's current condition. The information may be transmitted as a calls over land-line or internet-based telephone communication networks. Alternatively, the dialing station 8 may transmit the information over a cellular communications network. For example, the dialing station 8 can have the home telephone, cell phone, or other communication device numbers of the parents of the child, its grandparents, and other family members or caretakers. The dialing station 8 can also be programmed with the numbers for emergency services (EMS, Fire, Police, 911 etc.). When a distress condition is detected, the base unit 8 can call a plurality of the emergency contacts, which increases the chances of a rapid response to the distress condition of the infant. Further, the base unit 8 can also send the information via text messages or emails to the stored emergency contacts.

The dialing station 8 can have stored pre-recorded alert messages relating to the various conditions that are monitored by the sensor device. The pre-recorded messages are played when the emergency contacts are called. Thus, the base unit 8 can play a pre-recorded message related to the distress condition that is being sensed to the emergency contacts which not only alerts the emergency contact that there is a distress condition, but also the type of distress condition being sensed. The “pre-recorded” messages could also be in the form of preset text for messages when text messages and emails are sent out. The messages can be factory preset and come stored in the device. Additionally, the user can customize the messages to include individualized information. The individualized information can include the sensor user's name or special health conditions.

Further, the emergency contact persons can be provided with identification means, such as a RFID tag or an identification number. When one of the emergency contacts responds to the distress condition of the user, the emergency contact can provide the identification means to the dialing station 8 to indicate that one of the emergency contacts is responding to the distress condition. Then the dialing station 8 can alert the other emergency contacts and play a pre-recorded message indicating the identity of the emergency contact that is responding and that they are responding to the distress of the user. Thus, the other emergency contacts can be informed that someone is addressing the situation, which can alleviate some of the urgency of getting to the user by the other emergency contacts.

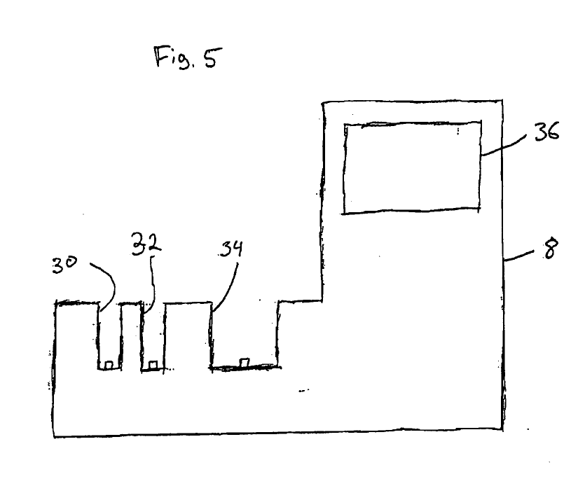

As shown in FIG. 5, the dialing station 8 can be in the form of a base unit. The dialing station 8 can be a base unit, which can be placed on a countertop or on a bedside table. The dialing station 8 includes docking ports 30, 32, and 34. The monitoring unit 2 can be docked in docking station 30 so that the monitoring unit 2 can be stored when not in use and the batteries can also be recharged. Further, the monitoring unit.2 and the docking station 30 may perform a synchronizing operation. For example, historical data and measure values can be stored and transmitted between the docking station 30 and the monitoring unit 2 during docking. A second docking station 32 may also be included for additional monitoring units 2, or it can be used to recharge a backup battery so that the monitoring unit 2 does not have to be placed in the docking station 30, but a battery could be swapped out. Additionally, a docking station 34 is included to recharge peripheral device 18, which is discussed below. The docking station 8 includes a display screen 36, which can display emergency messages and the vital signs of the user of the monitoring unit 2.

As shown in FIG. 4, a peripheral device 18 can also be included. The peripheral device 18 can be in the form of a small mobile device and can be roughly the size of a pager. The peripheral device 18 can receive alert messages from the dialing station 8 so that the user having the peripheral device 18 can be alerted and can monitor the status of the user of the monitoring unit 2. Also the monitoring unit 2 and the peripheral device 18 can include both long range and short range communication modules. Thus, they can both include a microphone and speaker (20 and 22 on the peripheral device 18) so the user of the monitoring unit 2 and the user of the peripheral device 18 can talk to each other. The short range communication can be accomplished with radio frequency signals, much like a walkie-talkie. The long range communication can be accomplished using cellular telephone, Wi-Fi, or other communication means, thus transmitting information wirelessly. The monitoring unit 2 and the peripheral device 18 may include cell phone communication capabilities, via for example a SIM chip. The peripheral device 18 includes a display screen 24, which can display emergency messages and the vital signs of the user of the monitoring unit 2.

In addition to the various sensor packages discussed above, the monitoring unit 2 can include additional features. As previously mentioned, the unit can include long and short range communication modules. Thus, the user can place calls and can communicate using microphone 14 and speaker 16. The user can use the unit to contact the emergency contact or emergency personnel. Further, the dialing can be voice or sound activated. Thus, if the user calls for “help,” “fire,” “police,” etc., the monitoring unit 2 will recognize these as voice commands and contact the appropriate contacts or emergency agencies in order to get assistance to the user. The sound activated contacting can also be initiated by a loud noise such as broken glass or a fall. The monitoring unit 2 may include cell phone communication capabilities, via, for example, a SIM chip. Thus, monitoring unit 2 can call/contact the emergency contact numbers via communication with cell phone transmission towers of a cellular communications network. The monitoring unit 2 can store the numbers of the emergency contacts and call them when the monitoring unit 2 indicates a distress condition in the user.

The monitoring unit 2 can optionally include a panic button 12. By holding down this button, emergency contacts or personnel can be alerted. Further, the monitoring unit can communicate with the base unit and other devices.

Further, the monitoring unit can store medical history, allergies, medication lists, reference electrocardiogram, emergency contacts, identification, and other information of the user. Thus, if the user is found and they are unable to communicate, emergency personal can obtain the user's information. This information can either be displayed on the unit's display screen 13 or can be downloaded to the emergency personnel computer systems. This can be transmitted for download either wired or wirelessly.

The monitoring unit can also function as a watch that displays time on the display screen 13.

Furthermore, the user's location may be sensed and determined, for example, within a house so that if a distress call is placed by the dialing station 8, not only the nature of the distress condition can be indicated, but also the current location of the user. The location and position of the user may be determined by providing various sensors 10 in rooms of a house, in particular the nursery for an infant, that communicate with the local positioning module located within the monitoring unit 2. Thus, the location of the user can be determined. Further, various areas of the house can be designated as restricted or “unsafe” zones, such as basements, garages, entrance and exit doors to the house, and workshop areas. If the user is located in a predetermined unsafe location, transmitters located within the sensors 10 will send a signal to the automated dialing station 8. The sensors can be proximity sensors that are placed in various rooms or can be placed in the threshold or doorways to these rooms or areas. The automated dialing station can either contact the emergency contacts or sound an alarm in the house. However, there are many different ways to track location and position and the invention is not so limited. For example, the monitoring unit 2 could utilize the Global Positioning System (GPS) or could emit Radio Frequency (RF) signals.

Further, the transmission unit can include GPS positioning capabilities, which can indicate the location of the user when outside of the house. Thus, the position of the user could be determined in an emergency situation.

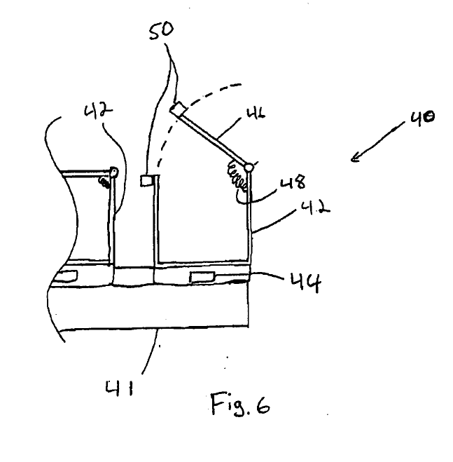

The monitoring system can also function as a reminder for the user, for example an elderly adult user, to take medications. An associated medication box 40 can be included to function with the monitoring unit 2 and the dialing station 8. The mediation box 40 can have seven individual cavities 42 per row and can have four rows. The medication box has a central control/processor unit in a base 41. Thus, the medication box 40 can be programmed with the user's medication schedule. As shown in FIG. 6, the medication box 40 can include for each cavity 42 a light element 44 (e.g., LEDs), a spring loaded hinge mechanism 48, and an electronically controlled latching mechanism 50. When it is the preprogrammed time for the user of the monitoring unit 2 to take a particular medication stored within a particular cavity 42, the control unit 41 causes the electronically controlled latching mechanism 50 to open and the spring loaded hinge mechanism 48 forces the lid 46 of the cavity to open. The electronically controlled latching mechanism 50 can be an electromagnetically controlled latch or other suitable device. Also, a light 44 beneath the cavity 42 is turned on, which causes the translucent cavity 42 to light up. This provides a visual signal that a medication should be taken and helps identify what particular cavity contains the medication that should be taken. In addition, the control unit 41 of the medication box 40 can send a signal to the monitoring unit 2 to indicate that it is time to take medication and the monitoring unit can alert the user. The monitoring unit 2 can also be programmed with the medication schedule and can alert the user without communication from the medication box 40.

Once the user takes the medication, the user closes the lid 46 of the cavity 42. The latching mechanism 50 includes a sensor, which can be a magnetic sensor, that indicates when the lid has be closed. Thus, the control unit can determine that the user has interacted with the medication box 40 and has taken the medication in the appropriate cavity 42 and the light 44 can be turned off However, if the lid remains open after a predetermined period of time, indicating that the medication has not been taken, the control unit 41 can send a signal to the dialing station 8. The dialing station 8 can then in turn contact the emergency contacts and/or the user of the peripheral device 18 that there is a problem and the user has not taken the medication.

As shown in FIG. 7, the monitoring unit 2 can be incorporated into a wrist cuff 60, that includes a portion that extends around the user's thumb or other fingers. The wrist cuff 60 can help to keep the monitoring unit 2 in place, especially when the user is a small infant with small wrists. By having a portion of the cuff 60 that extends around the user's thumb, the cuff 60 is held in place from sliding out of position. The wrist cuff 60 includes hook and loop type fastener strips, commonly known under the trade name Velcro®, that so that the cuff 60 can be wrapped around the wrist and held in place. Other types of fasteners, such as straps, belts, buckles, snaps, buttons, and other suitable fasteners can also be used. The wrist cuff 60 also provides additional contacting surface area with the user so that more area is provided for placing sensors on the user to monitor vital signs, including contact area over the wrist and the finger region of the user. As noted above, the monitoring unit can be coupled to any type of securing mechanism so that it may be secured to any body part that is readily accessible and convenient.

While the invention has been particularly shown and described with reference to preferred embodiments thereof, it will be understood by those skilled in the art that various changes in form and details may be made therein without departing from the spirit and scope of the invention.

We claim:

1. A monitoring device, comprising:
a plurality of sensors, each sensor configured to measure one of a plurality of vital sign parameters and a plurality of environmental parameters;
a memory configured to store at least a programmable upper limit and a programmable lower limit for each of the measured parameters;
a central processing unit configured to process the measured parameters and compare each measured parameter against its respective programmable upper limit and programmable lower limit; and
at least one communications module configured to transmit and receive data,
wherein the monitoring device is configured to automatically transmit an alert when one measured parameter is outside a range defined by its respective programmable upper limit and programmable lower limit.

2. The monitoring device as recited in claim 1, wherein the plurality of vital sign parameters include at least one of blood pressure, pulse rate, temperature, and oxygen saturation.

3. The monitoring device as recited in claim 1, wherein the plurality of environmental parameters include at least one of acceleration, temperature, pressure, humidity, carbon dioxide, and smoke.

4. The monitoring device as recited in claim 1, further comprising a panic button configured transmit information to alert a selected entity upon activation.

5. The monitoring device as recited in claim 1, further comprising a display configured to display data associated with at least one measured parameter.

6. The monitoring device as recited in claim 1, wherein the memory is configured to store at least one of medical history information and personal information.

7. The monitoring device as recited in claim 1, further comprising at least one of a microphone and a speaker.

8. The monitoring device as recited in claim 1, further comprising a positioning module configured to provide location information associated with the monitoring device.

9. The monitoring device as recited in claim 1, wherein the communications module is configured to transmit and receive data wirelessly.

10. The monitoring device as recited in claim 1, wherein a programmable upper limit and a programmable lower limit are customized to a user utilizing the monitoring device.

11. A monitoring system, comprising:
a monitoring unit including:
a plurality of sensors, each sensor configured to measure one of a plurality of vital sign parameter and a plurality of environmental parameter;
a memory configured to store at least a programmable upper limit and a programmable lower limit for each of the measured parameters;
a central processing unit configured to process the measured parameters and compare each of the measured parameters against its respective programmable upper limit and programmable lower limit; and
at least one monitoring unit communications module configured to transmit and receive data,
a base unit including:
a base unit communications module configured to transmit and receive data;
a processor configured to process data; and
a memory,
wherein the monitoring unit communicates data associated with the at least one measured parameter and is configured to automatically transmit an alert to the base unit when one measured parameter is outside a range defined by its respective programmable upper limit and programmable lower limit, and the base unit is configured to selectively transmit information based on at least one of the data associated with the at least one measured parameter and the alert.

12. The monitoring system as recited in claim 11, wherein the base unit communications module transmits the information via at least one of a cellular communications network and an Internet connection.

13. The monitoring system as recited in claim 11, wherein the memory is configured to store a plurality of contacts, each of the plurality of contacts having associated contact information.

14. The monitoring system as recited in claim 13, wherein the base unit is configured to recognize at least one identification module associated with at least one contact, and transmits data to the plurality of stored contacts indicating an identity of the recognized identification module.

15. The monitoring system as recited in claim 11, wherein the memory is configured to store at least one pre-recorded message, and wherein the processor is configured to select one of the at least one pre-recorded messages depending on at least one of the data associated with the at least one measured parameter and the alert, and the selected pre-recorded message is transmitted by the base communications unit.

16. The monitoring system as recited in claim 11, wherein the base unit includes a docking port configured to mate with the monitoring unit.

17. The monitoring system as recited in claim 11, further comprising a positioning module configured to provide location information associated with the monitoring unit.

18. The monitoring system as recited in claim 17, wherein the positioning module includes a plurality of proximity sensors.

19. The monitoring system as recited in claim 11, further comprising a peripheral mobile device having a mobile device communications module configured to communicate data with at least one of the base unit and the monitoring unit.

20. The monitoring system as recited in claim 11, further comprising a medication box,
wherein the monitoring unit is configured to provide a reminder corresponding to a medication schedule, and the medication box is configured to provide access to a medication according to the medication schedule and to transmit an alarm if the medication is not take according to the medication schedule.

21. A method for monitoring a user, comprising:
determining an upper limit and a lower limit to define a desired range for at least one of a plurality of vital sign parameters and a plurality of environmental parameters;
measuring the at least one parameter associated with the user;
comparing the measured at least one parameter to the respective desired range; and
automatically transmitting an alert when the measured parameter is outside the desired range defined by its respective upper limit and lower limit.
